(19) United States
(12) Patent Application Publication
(10) Pub. No.: US 2022/0346683 A1
(43) Pub. Date: Nov. 3, 2022

(54) INFORMATION PROCESSING SYSTEM AND INFORMATION PROCESSING METHOD

(71) Applicant: SONY GROUP CORPORATION, TOKYO (JP)

(72) Inventor: ATSUSHI SHIONOZAKI, TOKYO (JP)

(21) Appl. No.: 17/813,282

(22) Filed: Jul. 18, 2022

Related U.S. Application Data
(62) Division of application No. 15/745,777, filed on Jan. 18, 2018.

(30) Foreign Application Priority Data
Aug. 5, 2015 (JP) 2015-155300

(51) Int. Cl. A61B 5/16; G06F 16/9537; G06Q 30/02; A61B 5/00; A61B 5/11

(52) U.S. Cl. CPC A61B 5/165; G06F 16/9537; G06Q 30/0201; A61B 5/0022; A61B 5/1118; A61B 5/6801; A61B 5/1171

(57) ABSTRACT

To provide an information processing system and an information processing method that are capable of evaluating a specific space in association with feelings of users. The information processing system includes: an accumulation unit configured to accumulate variation in feelings of a user caused by going in and out of a specific space; and a control unit configured to calculate an evaluation value of the specific space on a basis of the variation in the feelings of the user.

This application is divisional application of U.S. patent application Ser. No. 15/745,777 filed on Jan. 18, 2018, which is a U.S. National Phase of International Patent Application No. PCT/JP2016/065711 filed on May 27, 2016, which claims priority benefit of Japanese Patent Application No. JP 2015-155300 filed in the Japan Patent Office on Aug. 5, 2015. Each of the above-referenced applications is hereby incorporated herein by reference in its entirety.

### TECHNICAL FIELD

The present disclosure relates to information processing systems and information processing methods.

### BACKGROUND ART

In recent years, technologies of evaluating feelings and psychological states of users have been proposed. For example, Patent Literature 1 listed below discloses a technology of sensing feelings of a user by using a communication device carried by a user who is joining an event. In addition, Patent Literature 2 listed below discloses a technology of analyzing sensor data detected by a sensor attached to a user in a shop and evaluating mental states of the user. In addition, Patent Literature 3 listed below discloses a technology of determining a degree of feelings of a user toward an object visually recognized by the user, from analysis of facial expression.

### CITATION LIST

### Patent Literature

Patent Literature 1: JP 2015-505702T
Patent Literature 2: JP 2013-537435T
Patent Literature 3: WO 2011/042989

### DISCLOSURE OF INVENTION

### Technical Problem

The above described technologies merely evaluate states of each user, and these technologies do not evaluate feelings of a user in association with spaces in a real world, for example. To use feeling information of users for various kinds of services, etc., it is necessary to evaluate spaces in association with feelings of users.

Accordingly, the present disclosure proposes a novel and improved information processing system and information processing method that are capable of evaluating a specific space in association with feelings of users.

### Solution to Problem

According to the present disclosure, there is provided an information processing system including: an accumulation unit configured to accumulate variation in feelings of a user caused by going in and out of a specific space; and a control unit configured to calculate an evaluation value of the specific space on a basis of the variation in the feelings of the user.

In addition, according to the present disclosure, there is provided an information processing system including: a communication unit configured to transmit space identification information indicating a specific space to an external device that is capable of calculating an evaluation value of the specific space, the evaluation value being based on variation in feelings of a user caused by going in and out of the specific space; and a control unit configured to generate a heat map image by mapping a pixel value representing the evaluation value on a position of the specific space on a basis of the evaluation value of the specific space indicated by the space identification information, the evaluation value being acquired by the external device via the communication unit.

In addition, according to the present disclosure, there is provided an information processing method including: accumulating variation in feelings of a user caused by going in and out of a specific space; and calculating, by a processor, an evaluation value of the specific space on a basis of the variation in the feelings of the user.

In addition, according to the present disclosure, there is provided an information processing method including: transmitting space identification information indicating a specific space to an external device that is capable of calculating an evaluation value of the specific space, the evaluation value being based on variation in feelings of a user caused by going in and out of the specific space; and generating, by a processor, a heat map image by mapping a pixel value representing the evaluation value on a position of the specific space on a basis of the evaluation value of the specific space indicated by the space identification information, the evaluation value being acquired by the external device.

### Advantageous Effects of Invention

As described above, according to the present disclosure, it is possible to evaluate spaces in association with feelings of users.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram illustrating an overview of an information processing system according to an embodiment of the present disclosure.

FIG. 2 is an explanatory diagram illustrating an overall configuration example of the information processing system according to the embodiment.

FIG. 3 is an explanatory diagram illustrating a configuration example of a space evaluation system according to the embodiment.

FIG. 4 is an explanatory diagram illustrating a configuration example of a heat map system according to the embodiment.

FIG. 5 is an explanatory diagram illustrating an example of a heat map image generated in the embodiment.

FIG. 6 is an explanatory diagram illustrating an example of a time-series image generated for a lifelog application generated in the embodiment.

FIG. 7 is an explanatory diagram illustrating an operation example of a happiness variation accumulation process according to the embodiment.

FIG. 8 is an explanatory diagram illustrating variation in states of data accumulated in an accumulation unit in the happiness variation accumulation process according to the embodiment.

FIG. 9 is an explanatory diagram illustrating an operation example of an average happiness variation value calculation process according to the embodiment.

FIG. 10 is an explanatory diagram illustrating variation in states of data accumulated in the accumulation unit in the average happiness variation value calculation process according to the embodiment.

FIG. 11 is an explanatory diagram illustrating an operation example of a Happiness Map generation process according to the embodiment.

FIG. 12 is an explanatory diagram illustrating an operation example of a My Happiness Map generation process according to the embodiment.

FIG. 13 is an explanatory diagram illustrating a second modification of the embodiment.

FIG. 14 is an explanatory diagram illustrating the second modification of the embodiment.

FIG. 15 is an explanatory diagram illustrating a hardware configuration of an information processing device according to the embodiment.

### MODE(S) FOR CARRYING OUT THE INVENTION

Hereinafter, (a) preferred embodiment(s) of the present disclosure will be described in detail with reference to the appended drawings. In this specification and the appended drawings, structural elements that have substantially the same function and structure are denoted with the same reference number, and repeated explanation of these structural elements is omitted.

Note that, in this description and the drawings, structural elements that have substantially the same function and structure are sometimes distinguished from each other using different alphabets after the same reference number. However, when there is no need in particular to distinguish structural elements that have substantially the same function and structure, the same reference number alone is attached.

Note that, the description is given in the following order.

### «1. Overview»

### «2. Configuration»

<2-1. Overall configuration>
<2-2. Configuration of space evaluation system>
<2-3. Configuration of heat map system>

### «3. Operation»

<3-1. Operation example of space evaluation>
<3-2. Operation example of heat map image generation>

### «4. Modification»

<4-1. First modification>
<4-2. Second modification>
<4-3. Third modification>
«5. Hardware configuration example»

### «6. Conclusion»

### 1. OVERVIEW

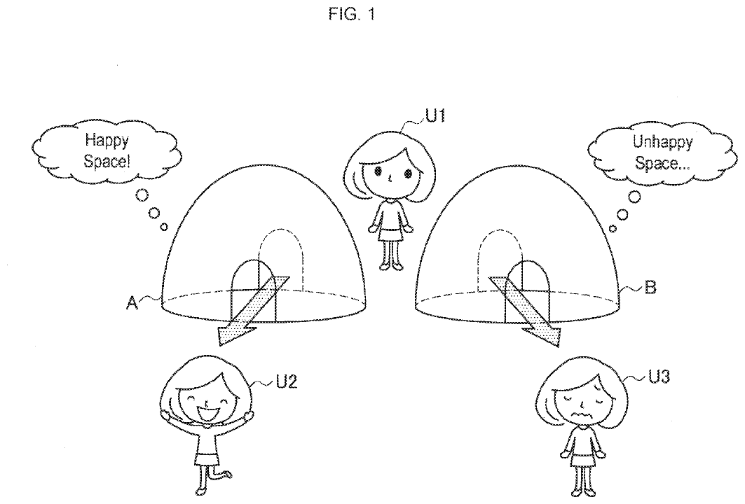

First, with reference to FIG. 1, an overview of an embodiment of the present disclosure will be described. An information processing system according to the embodiment measures a happiness level of a person (user) who uses (goes in and out of) a specific space where people go in and out (hereinafter, sometimes the specific space may be simply referred to as the space), and evaluates the space on the basis of the measured happiness level.

FIG. 1 is an explanatory diagram illustrating an overview of the information processing system according to the embodiment of the present disclosure. In FIG. 1, the information processing system according to the embodiment evaluates a space A as a space where a user feels happy in the case where a happiness level of the user after going out of the space A (state U2) is higher than a happiness level of the user before going into the space A (state U1). On the other hand, the information processing system according to the embodiment evaluates a space B as a space where a user feels unhappy in the case where a happiness level of the user after going out of the space B (state U3) is lower than a happiness level of the user before going into the space B (state U2).

It is believed that the user wants to go into a space where the user feels happy. Therefore, when the information processing system according to the embodiment provides evaluation information of a space where the user may feel happy, the user is capable of referring to this information to decide where to go, for example. In a similar way, when the information processing system according to the embodiment provides an owner of a space (such as a store) with evaluation information indicating a happiness level of the space, the owner is capable of using this information as an index for managing the space or for considering improvements in the space.

By using the evaluation index according to the embodiment, it is possible to measure how the space changes feelings (happiness level) of the user and evaluate the space. Therefore, the usage of the index is not limited to the usage described above, and it is possible to apply the evaluation index to various kinds of services or the like as the evaluation index that is directly linked to feelings of the user.

Note that, the specific space according to the embodiment may be a shop or a unit in a shopping mall. In addition, the specific space according to the present technology is not limited thereto. For example, the specific space may be any space with an entrance/exit such as a shopping mall as a whole, a large facility, a movie theater, an entertainment venue, or an event venue.

In addition, the entrance/exit of the space is not necessarily in contact with the space. For example, it is possible to consider an entrance/exit of a parking lot next to a shopping mall as an entrance/exit of the shopping mall. In addition, it is possible to calculate an evaluation value of the shopping mall on the basis of happiness levels acquired at the entrance/exit of the parking lot.

### 2. CONFIGURATION

The overview of the information processing system according to the embodiment of the present disclosure has been described above. Next, a configuration of the information processing system according to the embodiment will be described. Hereinafter, an overall configuration of the information processing system according to the embodiment will be described first, and then details of a space evaluation system and a heat map system of the information processing system according to the embodiment will be described in this order.

<2-1. Overall Configuration>

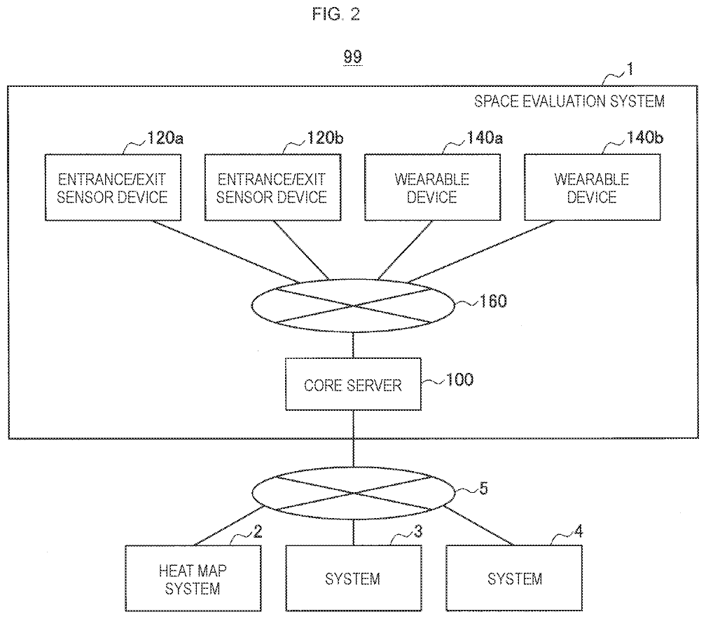

FIG. 2 is an explanatory diagram illustrating the overall configuration example of an information processing system 99 according to the embodiment. As illustrated in FIG. 2, the information processing system 99 according to the embodiment includes a space evaluation system 1, a heat map system 2, a system 3, a system 4, and a communication network 5, for example.

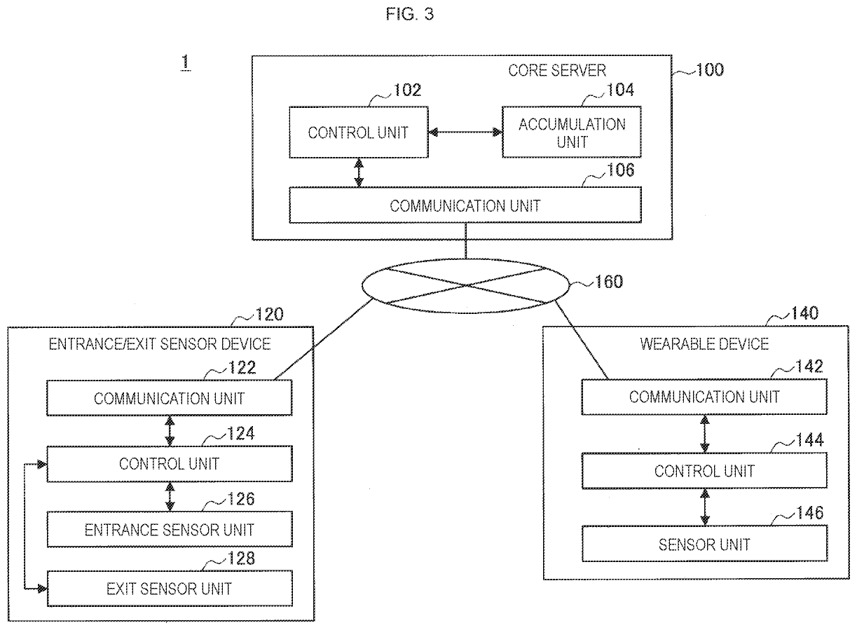

As illustrated in FIG. 2, the space evaluation system 1 is an information processing system including a core server 100, entrance/exit sensor devices 120a and 120b, wearable devices 140a and 140b, and a communication network 160. The core server 100 receives a happiness level of a user who has gone in and out of a specific space from the entrance/exit sensor device 120a, the entrance/exit sensor device 120b, the wearable device 140a, or the wearable device 140b via the communication network 160, and calculates an evaluation value of the specific space on the basis of variation in the happiness level. Note that, a detailed configuration of the space evaluation system 1 will be described later with reference to FIG. 3.

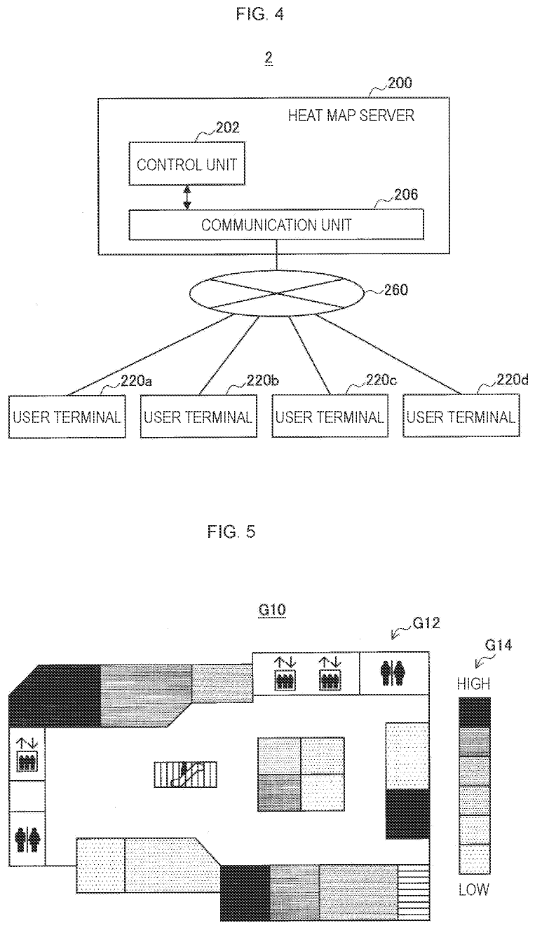

Each of the heat map system 2, the system 3, and the system 4 is an information processing system configured to receive the evaluation value of the specific space from the space evaluation system 1 and performs information processing using the evaluation value. For example, the heat map system 2 is an information processing system configured to generate a heat map image by mapping a pixel value representing the evaluation value (such as a value indicating color, brightness, or the like) of the space on a position of the specific space on the basis of the evaluation value. Note that, the configuration of the heat map system 2 will be described later with reference to FIGS. 4 to 6. In addition, an example of information processing using the evaluation value other than the generation of the heat map image (such as examples of information processing carried out by the system 3 and the system 4) will be described later as a third modification.

The communication network 5 is a wired or wireless communication channel through which information is transmitted from devices or systems connected with the communication network 5. For example, the communication network 5 may include a public network, various kinds of local area networks (LANs), a wide area network (WAN), and the like. The public network includes the Internet, a satellite communication network, a telephone network, and the like, and the LANs include Ethernet (registered trademark). In addition, the communication network 5 may include a dedicated line network such as an Internet Protocol Virtual Private Network (IP-VPN).

<2-2. Configuration of Space Evaluation System>

The overall configuration example of the information processing system 99 according to the embodiment has been described above. Next, with reference to FIG. 3, a configuration example of the space evaluation system 1 according to the embodiment will be described. FIG. 3 is an explanatory diagram illustrating a configuration example of the space evaluation system 1 according to the embodiment. As illustrated in FIG. 3, the space evaluation system 1 according to the embodiment is an information processing system including the core server 100, the entrance/exit sensor device 120, the wearable device 140, and the communication network 160.

Although FIG. 3 illustrates the single entrance/exit sensor device 120 and the single wearable device 140, the space evaluation system according to the embodiment may include a plurality of the entrance/exit sensor devices and a plurality of the wearable devices as illustrated in FIG. 2, for example. For example, the number of the entrance/exit sensor devices may be equal to the number of specific spaces that are evaluation targets of the space evaluation system 1 (such as the number of shops in a shopping mall) or may be equal to the number of entrances/exits of a specific space, and the number of the wearable devices may be equal to the number of users. In addition, the space evaluation system according to the embodiment may be an information processing system including only one of the entrance/exit sensor device and the wearable device.

Note that, the configuration of the communication network 160 is substantially the same as the configuration of the communication network 5 described with reference to FIG. 2. Therefore, the description of the communication network 160 is omitted. Next, configurations of the core server 100, the entrance/exit sensor device 120, and the wearable device 140 of the space evaluation system 1 according to the embodiment will be described in this order.

(Core Server)

As illustrated in FIG. 3, the core server 100 is an information processing device including a control unit 102, an accumulation unit 104, and a communication unit 106. The core server 100 receives a happiness level of a user who has gone in and out of a specific space from the entrance/exit sensor device 120 or the wearable device 140 via the communication network 160, and calculates an evaluation value of the specific space on the basis of variation in the happiness level.

The control unit 102 controls the core server 100 as a whole. For example, the control unit 102 controls the accumulation unit 104 (to be described later) such that the accumulation unit 104 accumulates or acquires data. In addition, the control unit 102 controls communication (transmission or reception) performed by the communication unit 106, for example.

For example, via the communication unit 106, the control unit 102 acquires feeling data of each user who has gone in and out of a specific space and causes the accumulation unit 104 to accumulate the feeling data. In addition, the control unit 102 calculates variation in feelings of each user caused by going in and out of the specific space on the basis of the feeling data, and causes the accumulation unit 104 to accumulate the variation in the feelings. For example, the control unit 102 may calculate the variation in the feelings of each user on the basis of difference between feeling data obtained when each user goes out of a specific space and feeling data obtained when each user goes into the specific space.

Note that, the feeling data according to the embodiment may be a happiness level. For example, the control unit 102 may calculate the variation in the feelings (happiness variation) of each user on the basis of difference between a happiness level obtained when each user goes out of a specific space and a happiness level obtained when each user goes into the specific space. Note that, a timing when the user goes into a specific space may be a timing immediately before the user goes into the specific space or may be a timing immediately after the user goes into the specific space. Similarly, a timing when the user goes out of a specific space may be a timing immediately before the user goes out of the specific space or may be a timing immediately after the user goes out of the specific space.

Happiness variation H_{δ} of a user is represented by the following equation, where Hₜ represents a happiness level obtained when the user goes into a specific space, and H_{t+dw} represents a happiness level obtained when the user goes out of the specific space (dw represents dwell time).

H_{δ}=H_{t+dw}−Hₜ [Math. 1]

In the case where the happiness variation H_{δ} is positive (H_{δ}>0), the specific space is considered as a space whose happiness level increases when the user goes in and out of the specific space (a space where the user feels happy). Alternatively, in the case where the happiness variation H_{δ} is negative (H_{δ}<0), the specific space is considered as a space whose happiness level decreases when the user goes in and out of the specific space. Alternatively, in the case where the happiness variation H_{δ} is zero (H_{δ}=0), the specific space is considered as a space whose happiness level does not vary (whose happiness level is constant) when the user goes in and out of the specific space.

In addition, the control unit 102 calculates an evaluation value of the specific space on the basis of the variation in feelings of the user (happiness variation in the embodiment) that is caused by going in and out of the specific space and that is accumulated in the accumulation unit 104, and causes the accumulation unit 104 to accumulate it. For example, the control unit 102 may calculate an average value of happiness variation of a user who has gone in and out of the specific space in a predetermined time period (such as in a day), and may use the average value (an average of happiness variation of the user during the predetermined time period) as an evaluation value.

Note that, the evaluation value calculated by the control unit 102 is not limited thereto. For example, the control unit 102 may sum an average of happiness variation of a user for each day (predetermined time period) during a certain number of days (total days), and divide the summed averages by the total days to obtain an average happiness variation value as the evaluation value. Alternatively, the control unit 102 may use happiness variation of a specific user as an evaluation value.

Note that, the feeling data used for calculating the evaluation value may be acquired on the basis of user information detected by an entrance sensor unit 126 and an exit sensor unit 128 included in the entrance/exit sensor device 120 installed such that the entrance/exit sensor device 120 is capable of detecting information of an entrance/exit of a specific space. By using such structural elements, it is possible to acquire feeling data of a user who does not carry a sensing device or the like. Note that, the entrance/exit sensor device 120 and the user information detected by the entrance sensor unit 126 and the exit sensor unit 128 of the entrance/exit sensor device 120 will be described later.

Alternatively, the feeling data used by the control unit 102 for calculating the evaluation value may be acquired on the basis of biological information of a user detected by a sensor unit 146 of the wearable device 140 attached to the user. By using such a structural element, it is possible to acquire feeling data of a user with regard to a space where it is difficult to install the entrance/exit sensor device 120. Note that, the wearable device 140 and the biological information of the user detected by the sensor unit 146 of the wearable device 140 will be described later.

In addition, in the case where the communication unit 106 receives space identification information indicating a specific space from an external device (device outside of the space evaluation system 1), the control unit 102 returns an evaluation value of the specific space indicated by the space identification information to the external device via the communication unit 106. Alternatively, in the case where the communication unit 106 receives space identification information and user identification information (such as a user ID) indicating a specific user from an external device, the control unit 102 returns an evaluation value that is an evaluation of the specific space made by the specific user to the external device via the communication unit 106. The specific user is indicated by the user identification information, and the specific space is indicated by the space identification information.

Note that, the external device configured to transmit space identification information indicating a specific space to the core server 100 may be a device included in the heat map system 2, the system 3, or the system 4 described with reference to FIG. 2, for example. In addition, for example, in the case where the specific space is a shop, the space identification information may be a unique ID (shop ID) allocated to each shop.

According to such structural elements, it is possible for the space evaluation system 1 to cooperate with the external device and an external system, and it is possible for the external device and the external system to provide various kinds of services and applications to users or an owner of a specific space.

Under the control of the control unit 102, the accumulation unit 104 accumulates various kinds of data and provides the various kinds of accumulated data to the control unit 102. For example, the accumulation unit 104 accumulates feeling data (such as a happiness level) of each user who has gone in and out of a specific space, variation in feelings of a user (such as happiness variation) caused by going in and out of the specific space, and the evaluation value (such as average happiness variation value).

The communication unit 106 communicates with devices in the space evaluation system 1 and devices (external devices) outside of the space evaluation system 1. For example, the communication unit 106 receives space identification information indicating a specific space from the external device. In addition, the communication unit 106 accepts the control of the control unit 102 and transmits an evaluation value of the specific indicated by the space identification information to the external device. In addition, the communication unit 106 receives a happiness level (feeling data) form the entrance/exit sensor device 120 and the wearable device 140.

(Entrance/exit sensor device) The configuration of the core server 100 has been described above. Next, a configuration of the entrance/exit sensor device 120 will be described. As illustrated in FIG. 3, the entrance/exit sensor device 120 is an information processing device including a communication unit 122, a control unit 124, an entrance sensor unit 126, and an exit sensor unit 128. The entrance/exit sensor device 120 may be installed (for example, at a position near entrance/exit) such that the entrance sensor unit 126 and the exit sensor unit 128 are capable of detecting information regarding an entrance/exit of a specific space.

The communication unit 122 communicates with the core server 100 via the communication network 160. For example, the communication unit 122 transmits feeling data (such as happiness level) to the core server 100 in addition to the space identification information (such as a shop ID), user identification information (user ID), acquisition date/time, and a result of detecting going in and out of a space. Note that, it is possible to set a unique ID (space ID or shop ID) for each entrance/exit sensor device in advance, as space identification information. In addition, the user identification information, the acquisition date/time, the result of detecting going in and out of a space, and the feeling data are provided to the communication unit 122 by the control unit 124 (to be described later).

The control unit 124 controls the entrance/exit sensor device 120 as a whole. For example, the control unit 124 acquires feeling data on the basis of information regarding a user (user information) detected by the entrance sensor unit 126 and the exit sensor unit 128, and controls the communication unit 122 such that the communication unit 122 transmits the feeling data to the core server 100. In addition, the control unit 124 identifies the user on the basis of the user information detected by the entrance sensor unit 126 and the exit sensor unit 128.

Next, a feeling data acquisition example will be described in the case where the entrance sensor unit 126 and the exit sensor unit 128 are cameras capable of acquiring images and detecting information (such as faces) on users (people) in the images. Note that, the entrance sensor unit 126 and the exit sensor unit 128 included in the entrance/exit sensor device 120 are not limited to the cameras. The entrance sensor unit 126 and the exit sensor unit 128 may be other sensors as long as the sensors are capable of detecting user information from which the control unit 124 is capable of acquiring feeling data and identifying the user. Configurations of the entrance sensor unit 126 and the exit sensor unit 128 according to the embodiment will be described later.

On the basis of user information detected by the entrance sensor unit 126 and the exit sensor unit 128, the control unit 124 acquires feeling data (happiness level) obtained when each user goes into a specific space and feeling data (happiness level) obtained when each user goes out of the specific space. The feeling data may be included in the user information, or may be obtained through calculation performed by the control unit 124 on the basis of the user information.

For example, the control unit 124 may recognize a person (user) and facial expressions of the person from the images captured by the entrance sensor unit 126 and the exit sensor unit 128 to acquire the feeling data. For example, the control unit 124 may recognize a person's smile, evaluate a smile level, and acquire the smile level as the happiness level. In addition, the control unit 124 may determine a happiness level on the basis of whether the recognized person is with another person. For example, in the case where there is another person near the recognized person, a high happiness level may be set. On the other hand, the control unit 124 may discriminate age and sex of the other person near the recognized person, determine an attribute of a group including the recognized person (such as a couple, a family, friends, or the like), and specify a happiness level in accordance with the attribute.

In addition, the control unit 124 identifies the user on the basis of the user information detected by the entrance sensor unit 126 and the exit sensor unit 128. If detected user information is information regarding a new user (user without set user identification information), the control unit 124 sets user identification information (user ID) unique to the user. For example, the control unit 124 according to the embodiment may identify a user through a face recognition technology by using information regarding a face detected by the entrance sensor unit 126 and the exit sensor unit 128.

In addition, the control unit 124 provides the communication unit 122 with the acquired feeling data, the acquired user identification information, and the acquisition date/time (date/time when the control unit 124 acquires the user information from the entrance sensor unit 126 or the exit sensor unit 128).

Note that, the control unit 124 determines whether the acquired feeling data (happiness level) is feeling data obtained when the user goes into a space or feeling data obtained when the user goes out of the space, and provides the determination result (a result of determining going in and out of the space) to the communication unit 122. For example, in the case of acquiring feeling data on the basis of user information detected by the entrance sensor unit 126, the control unit 124 may determine that the feeling data is feeling data obtained when the user goes into the space. Alternatively, in the case of acquiring feeling data on the basis of user information detected by the exit sensor unit 128, the control unit 124 may determine that the feeling data is feeling data obtained when the user goes out of the space.

The entrance sensor unit 126 is a sensor installed to acquire information regarding an entrance of a specific space. For example, the entrance sensor unit 126 may be a camera that is installed at a position and angle capable of capturing an image of a user going into the specific space through the entrance, acquires the captured image, and detects information (such as a face) of the user (person) in the image. Note that, in the case where the entrance also serves as the exit, the entrance sensor unit 126 may be installed at a position and angle capable of capturing images of users going into the space.

The exit sensor unit 128 is a sensor installed to acquire information regarding an entrance of a specific space. For example, the exit sensor unit 128 may be a camera that is installed at a position and angle capable of capturing an image of a user going out of the specific space through the exit, acquires the captured image, and detects information (such as a face) of the user (person) in the image. Note that, in the case where the exit also serves as the entrance, the exit sensor unit 128 may be installed at a position and angle capable of capturing images of users going out of the space.

Although, FIG. 3 illustrates an example in which the entrance/exit sensor device 120 includes one entrance sensor unit and one exit sensor unit, it is also possible for the entrance/exit sensor device 120 to include a plurality of entrance sensor units and a plurality of exit sensor units. For example, the number of the entrance sensor units of the entrance/exit sensor device 120 may be equal to the number of entrances, and the number of the exit sensor units of the entrance/exit sensor device 120 may be equal to the number of exits. In addition, the entrance sensor unit and the exit sensor unit may be independent. For example, each of the entrance sensor unit and the exit sensor unit may be a sensor device configured to provide (transmit) detected information to a device including the control unit 124.

(Wearable Device)

The configuration of the entrance/exit sensor device 120 has been described above. Next, a configuration of the entrance/exit sensor device 120 will be described. As illustrated in FIG. 3, the wearable device 140 is an information processing device including a communication unit 142, a control unit 144, and a sensor unit 146. The wearable device 140 is a device configured to acquire biological information of a user going in and out of a specific space and provides the biological information to the core server 100. For example, the wearable device 140 may be attached to the user.

The communication unit 142 communicates with the core server 100 via the communication network 160. For example, the communication unit 122 transmits feeling data (such as happiness level) to the core server 100 in addition to the space identification information (such as a shop ID), user identification information (user ID), and acquisition date/time. Note that, the space identification information may be acquired on the basis of a beacon signal received by the communication unit 142 from a beacon transmission device (not illustrated). The beacon transmission device is installed in each space or in the vicinity of each space. In addition, in the case where the wearable device 140 is capable of acquiring position information, the space identification information may be acquired on the basis of the position information. In addition, an ID (user ID) unique to each wearable device may be set in advance as the user identification information. In addition, acquisition date/time and feeling data are provided to the communication unit 122 by the control unit 124 (to be described later).

The control unit 144 controls the wearable device 140 as a whole. For example, the control unit 144 acquires feeling data on the basis of user biological information (such as blood flow, heart rate, body temperature, brain waves, or voice) detected by the sensor unit 146, and controls the communication unit 142 such that the communication unit 142 transmits the feeling data to the core server 100.

The sensor unit 146 is a sensor configured to acquire biological information of a user. For example, the sensor unit 146 may include a blood flow sensor, a heart rate sensor, a body temperature sensor, a brain wave sensor, a microphone, or the like to acquire blood flow, a heart rate, body temperature, brain waves, voice, or the like of the user.

<2-3. Configuration of Heat Map System>

The configuration example of the space evaluation system 1 according to the embodiment has been described above. Next, with reference to FIG. 4, a configuration example of the heat map system 2 according to the embodiment will be described. FIG. 4 is an explanatory diagram illustrating the configuration example of the heat map system 2 according to the embodiment. As illustrated in FIG. 4, the heat map system 2 according to the embodiment is an information processing system including a heat map server 200, user terminals 220a to 220d, and a communication network 260.

Note that, FIG. 4 illustrates the example in which the heat map system 2 includes four user terminals 220a to 220d. However, the number of user terminals of the heat map system 2 may be more than or less than four (may be one). Note that, the configuration of the communication network 260 is substantially the same as the configuration of the communication network 5 described with reference to FIG. 2. Therefore, the description of the communication network 160 is omitted.

(Heat Map Server)

As illustrated in FIG. 4, the heat map server 200 is an information processing device including a control unit 202 and a communication unit 206.

The control unit 202 controls communication performed by the communication unit 206, and generates a heat map image by mapping a pixel value representing an evaluation value of a specific space on a position of the specific space, on the basis of the evaluation value. The evaluation value of the specific space is acquired by the external device (core server 100 of the space evaluation system 1 according to the embodiment) via the communication unit 206 (to be described later). For example, when the communication unit 206 transmits space identification information (such as a shop ID) indicating a specific space to the core server 100, the core server 100 returns an evaluation value of the specific space indicated by the space identification information to the communication unit 206 of the heat map server 200. The evaluation value of the specific space indicated by the space identification information may be an average of user happiness variation during a predetermined time period, for example. In such a case, a heat map image generated by the control unit 202 is a heat map image from which it is possible to recognize an average of user happiness variation in each space. Hereinafter, sometimes such a heat map image may be referred to as a Happiness Map.

For example, the control unit 202 may normalize evaluation values on the basis of the evaluation values of a plurality of specific spaces, and then may decide pixel values representing the evaluation values in a heat map image. In addition, the control unit 202 may decide the pixel values such that brightness and colors differ from each other in accordance with the pixel values. For example, in a heat map image generated by the control unit 202, a high brightness pixel value may be mapped on a position of a space with a high evaluation value, and a low brightness pixel value may be mapped on a position of a space with a low evaluation value. Alternatively, in a heat map image generated by the control unit 202, a long wavelength pixel value (such as red) may be mapped on a position of a space with a high evaluation value, and a short wavelength pixel value (such as blue) may be mapped on a position of a space with a low evaluation value.

According to such a configuration, it is possible for a user to visually recognize a space where the user is likely to feel happy by seeing the heat map image. For example, the heat map image is referred to as an index for deciding whether to go into a shop.

FIG. 5 is an explanatory diagram illustrating an example of the heat map image generated by the control unit 202. As illustrated in FIG. 5, the heat map image G10 includes a floor map G12 of a shopping mall in which pixel values indicating evaluation values of respective spaces are mapped on positions of the respective spaces (positions of respective shops in the shopping mall in the example illustrated in FIG. 5). In addition, as illustrated in FIG. 5, the heat map image G10 may include a legend G14 indicating a correspondence between evaluation values and pixel values.

Note that, as described later, the heat map image related to the evaluation values of the spaces (shops) in the shopping mall as illustrated in FIG. 5 may be displayed on the user terminals 220a to 220d carried by users or may be displayed on a display device or the like (not illustrated) installed at an entrance or the like of the shopping mall.

In addition, the control unit 202 may transmit the space identification information (such as a shop ID) indicating a specific space and user identification information (such as a user ID) indicating a specific user to the core server 100 (external device) via the communication unit 206. In addition, the control unit 202 may acquire an evaluation value of a specific space from the core server 100 via the communication unit 206. The specific space is indicated by space identification information, and the evaluation value is evaluation made by a specific user indicated by the user identification information. Here, the evaluation value of the specific space that is evaluation made by the specific user indicated by the user identification information may be happiness variation of the specific user with regard to the space. In addition, the control unit 202 may generate a heat map image for the specific user by mapping a pixel value representing the evaluation value on the position of the specific space on the basis of the evaluation value acquired from the core server 100. In such a case, the heat map image generated by the control unit 202 is a heat map image from which it is possible to recognize happiness variation of the user for each space. Hereinafter, sometimes such a heat map image for a specific user that is personalized for the specific user may be referred to as a My Happiness Map.

As described above, to create a heat map image for a specific user, it is only necessary to track people recognized in respective spaces as the same person (on the basis of a single user ID). For example, in the space evaluation system 1, it is possible to check whether people in different spaces are the same person by using a face recognition technology (face authentication technology) or the like, or it is possible to recognize pieces of information obtained from a single wearable device as information regarding a single person.

In addition, pixel values representing evaluation values may be mapped not only on a specific facility map or the like but also on any map. Specifically, the heat map image for a specific user may be used while mapped on any map depending on the user. In the case where pixel values representing evaluation values are mapped on any map as described above, sometimes it is difficult to map pixel values on specific spaces (such as shops or facilities) on one-to-one basis, depending on zoom levels (enlargement ratio for displaying the map). In such a case, the control unit 202 may aggregate evaluation values of a plurality of spaces (such as shops or facilities) within a predetermined range (for example, decide a pixel value by averaging evaluation values of the plurality of spaces), and generate a heat map image, for example.

According to such a configuration, it is possible to obtain a heat map image (My Happiness Map) of happiness levels for each user. Therefore, it is possible for a user to find a place where the user is likely to feel happy and use such information as a reference for deciding a future destination, for example. In addition, it is possible to create an application or the like configured to notify of recommendation information for recommending a user to visit a space with a high evaluation value made by the user (happiness variation) again on the basis of the My Happiness Map. In addition, it is possible to tell family members or friends about a space where a user feels happy by sharing the user's My Happiness Map with the family members or friends through a social network service (SNS), an application, or the like.

In addition, the control unit 202 generates an image in which pixel values representing evaluation values that are evaluation made by a specific user with regard to a space visited by the specific user are arranged in chronological order. For example, the control unit 202 may generate a time-series image in which a pixel value representing an evaluation value corresponding to a first visited space and a pixel value representing an evaluation value corresponding to a second visited space are arranged in chronological order. According to such a configuration, it is possible for a lifelog application configured to track behavior and the like of a user to record and present variation in feelings of the user in addition to information indicating the Five W's (behavior history or the like based on position information, time, photograph, and sensor information).

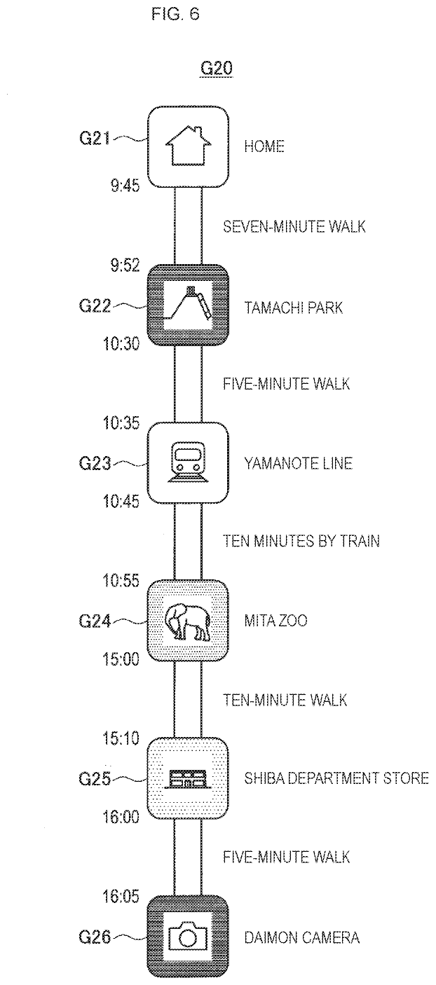

FIG. 6 is an explanatory diagram illustrating an example of the time-series image generated by the control unit 202 for the lifelog application. As illustrated in FIG. 6, in a time-series image G20, icons G21 to G26 are arranged in chronological order. The icons G21 to G26 indicate spaces visited by a specific user, transfer pathways of the specific user, and a like in a day. Among the icons G21 to G26, pixel values representing evaluation values of specific spaces are mapped on the icons G22 and G24 to G26 corresponding to the specific spaces that are targets of evaluation made by the space evaluation system 1. Note that, in the example of the time-series image illustrated in FIG. 6, the evaluation values are variation in happiness of the user in the respective spaces, and relations between the evaluation values and the pixel values are similar to the example of the heat map image illustrated in FIG. 5.

In the example illustrated in FIG. 6, a happiness level in the Tamachi park (icon G22) where a user has played with children significantly increases in comparison to the other places, and the largest increase in a happiness level in that day is recorded (icon G24) with regard to the Mita zoo visited subsequently. Note that, although the user has enjoyed shopping in the Shiba department store (icon G25), a happiness level increases slightly in comparison with the Tamachi park or the Mita zoo. In the Daimon Camera (camera retail store) (icon G26) the user has visited last in that day, a happiness level significantly increases again in comparison with the other places since the user has spent time on personal interests.

The communication unit 206 illustrated in FIG. 4 communicates with the user terminals 220a to 220d via the communication network 260 under the control of the control unit 202. In addition, under the control of the control unit 202, the communication unit 206 communicates with the core server 100 of the space evaluation system 1 via the communication network 5 illustrated in FIG. 2.

For example, the communication unit 206 transmits space identification information indicating a specific space to an external device (core server 100 according to the embodiment) that is capable of calculating an evaluation value of the specific space on the basis of variation in feelings of a user caused by going in and out of the specific space. In addition, the communication unit 206 transmits an image generated by the control unit 202 (such as the heat map image or the time-series image) to the user terminals 220a to 220d.

(User Terminal)

The user terminals 220a to 220d are each a device configured to receive an image generated by the heat map server 200 (such as the heat map image or the time-series image) from the heat map server 200 via the communication network 260, and display the image. For example, the user terminals 200a to 200d may be carried by users. Specifically, the user terminals 200a to 200d may be carried by users indicated by user identification information transmitted from the heat map server 200 to the core server 100.

### 3. OPERATION

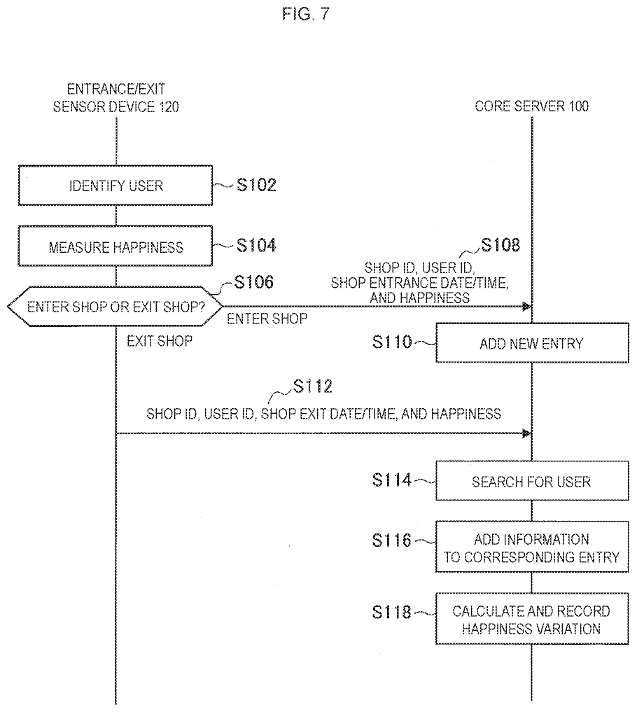

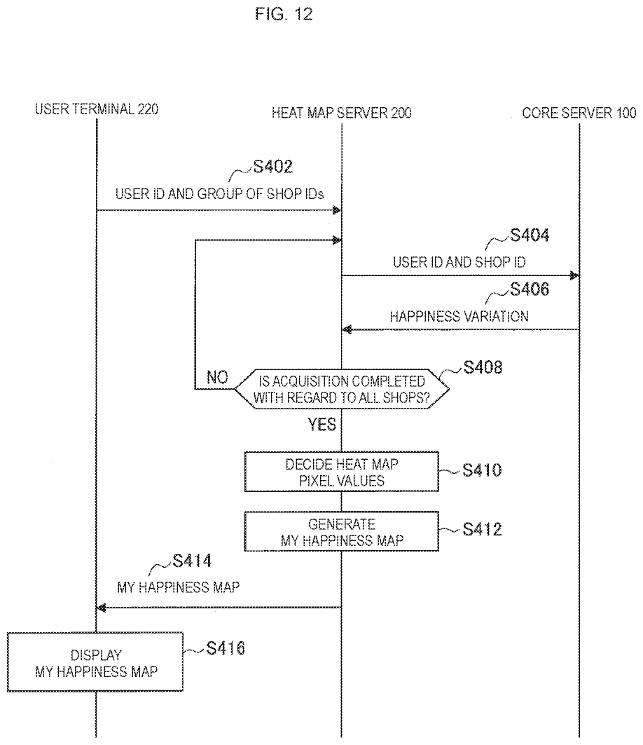

The configuration of the information processing system 99 according to the embodiment has been described above. Next, operation performed by the information processing system 99 according to the embodiment will be described. Hereinafter, an operation example of space evaluation made by the space evaluation system 1 will be described with reference to FIG. 7 to FIG. 10, and then an operation example of heat map image generation performed by the space evaluation system 1 and the heat map system 2 will be described with reference to FIG. 11 and FIG. 12. Note that, an example of a case where a specific space according to the embodiment is a shop in a shopping mall or the like will be described.

<3-1. Operation Example of Space Evaluation>

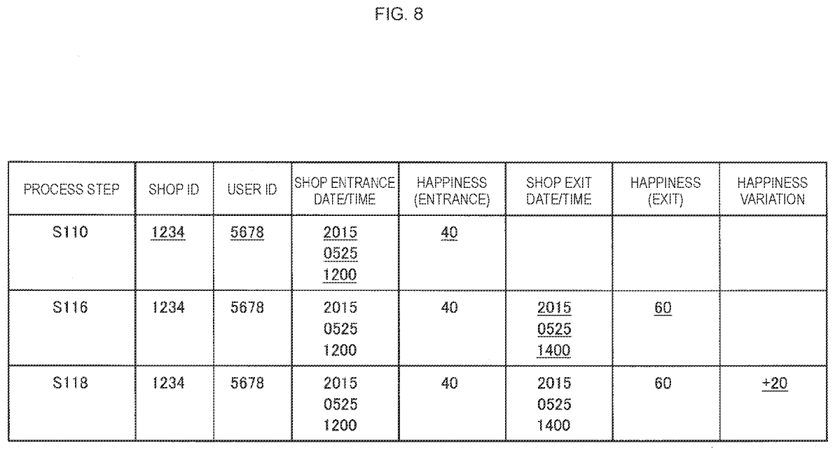

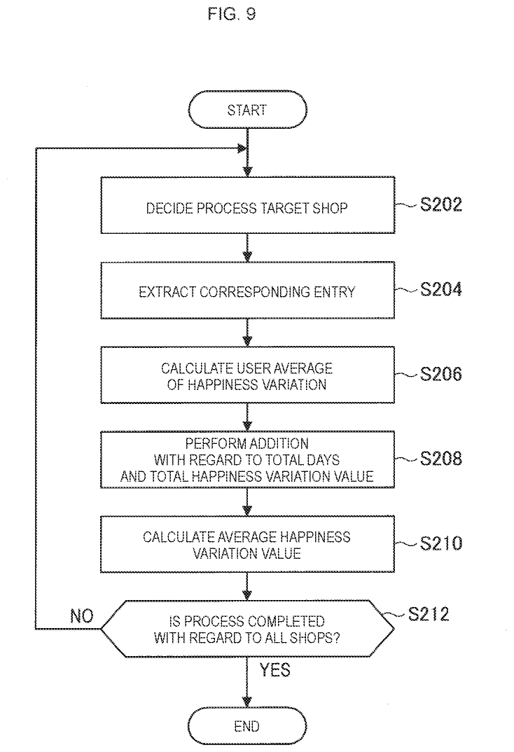

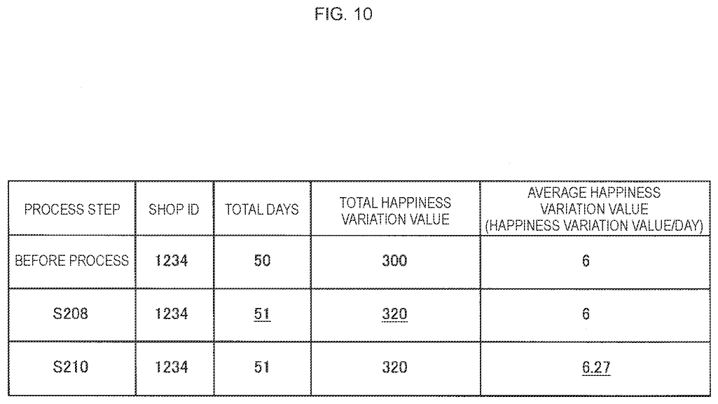

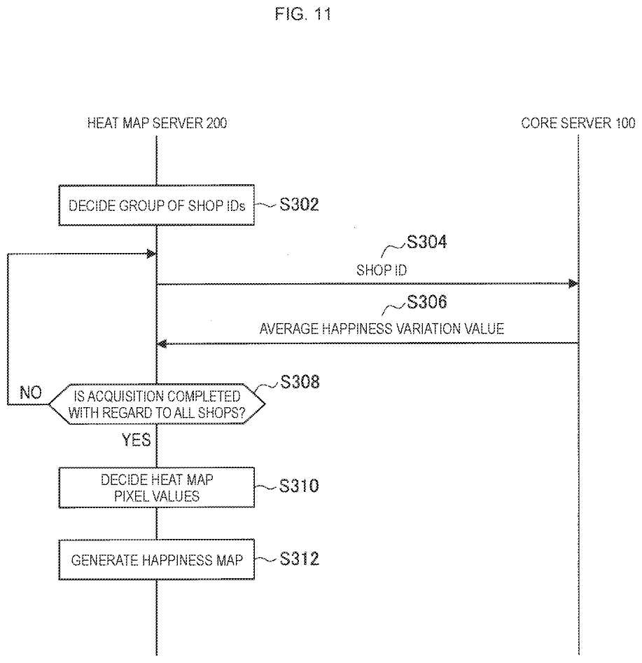

The space evaluation system 1 according to the embodiment makes space evaluation by performing a happiness variation accumulation process and an average happiness variation value calculation process. The happiness variation accumulation process is performed through detection of user information, and the average happiness variation value calculation process is performed for each predetermined time period (such as for each day). Hereinafter, the happiness variation accumulation process will be described with reference to FIG. 7 and FIG. 8, and then the average happiness variation value calculation process will be described with reference to FIG. 9 and FIG. 10.

(Happiness Variation Accumulation Process)

FIG. 7 is an explanatory diagram illustrating an operation example of the happiness variation accumulation process according to the embodiment. In addition, FIG. 8 is an explanatory diagram illustrating variation in states of data accumulated in the accumulation unit 104 in the happiness variation accumulation process according to the embodiment. Note that, in FIG. 8, information added in each process step is underlined.

Note that, an example will be described in which the entrance/exit sensor device 120 acquires information (happiness level) and provides the information to the core server 100. However, space evaluation can be made in a similar way even in the case where the wearable device 140 acquires and provides information.

First, as illustrated in FIG. 7, when a user is detected through sensing, the entrance/exit sensor device 120 identifies the user on the basis of user information of the detected user (S102) and measures (acquires) a happiness level (S104). Next, the entrance/exit sensor device 120 determines whether the happiness level is acquired when the user goes into a shop (shop entrance timing) or when the user goes out of the shop (shot exit timing) (S106).

In the case where the happiness level is acquired at shop entrance timing (“ENTER SHOP” in Step S106), the entrance/exit sensor device 120 transmits a shop ID (space identification information), a user ID (user identification information), shop entrance date/time (acquisition date/time), and a happiness level to the core server 100 (S108). Note that, the shop entrance date/time may include information regarding a determination result indicating that the happiness level transmitted at the same time is acquired at the shop entrance timing. The core server 100 that has received the shop ID, the user ID, the shop entrance date/time, and the happiness level acquired at the shop entrance timing from the entrance/exit sensor device 120 adds a new entry as illustrated in a row of the process step S110 in FIG. 8, and causes the accumulation unit 104 to accumulate it (S110).

On the other hand, in the case where the happiness level is acquired at shop exiting timing (“EXIT SHOP” in Step S106), the entrance/exit sensor device 120 transmits a shop ID (space identification information), a user ID (user identification information), shop exit date/time (acquisition date/time), and a happiness level to the core server 100 (S112). Note that, the shop exit date/time may include information regarding a determination result indicating that the happiness level transmitted at the same time is acquired at the shop exit timing. The core server 100 that has received the shop ID, the user ID, the shop exit date/time, and the happiness level from the entrance/exit sensor device 120 searches the accumulated information for an appropriate user by using the shop ID and the user ID (S114).

Next, as shown in a row of a process step S116 in FIG. 8, the core server 100 adds information regarding the shop exit dates/times and happiness levels acquired at the shop exit timings to the entry obtained through the user search (entry added in Step S110 when the user goes into the shop) (S116).

Next, the core server 100 calculates happiness variation as variation in feelings of the user caused by going in and out of the specific space, and the accumulation unit 104 of the core server 100 accumulates (records) the happiness variation in the corresponding entry (S118). For example, in the example illustrated in FIG. 8, the happiness level acquired at the shop entrance timing is 40, and the happiness level acquired at the shop exit timing is 60. Therefore, as shown in the row of the process step S118, the happiness variation of +20 is recorded.

(Average Happiness Variation Value Calculation Process)

The operation example of the happiness variation accumulation process has been described above. Next, an operation example of the average happiness variation value calculation process according to the embodiment will be described. The average happiness variation value calculation process according to the embodiment is performed by the core server 100 for each predetermined time period (for each day according to this operation example). FIG. 9 is an explanatory diagram illustrating the operation example of the average happiness variation value calculation process according to the embodiment. In addition, FIG. 10 is an explanatory diagram illustrating variation in states of data accumulated in the accumulation unit 104 with regard to average happiness variation values (example of evaluation values) in a certain shop during the average happiness variation value calculation process according to the embodiment. Note that, in FIG. 10, a row before performing the process step shows a state of data of a shop before performing the average happiness variation value calculation process on the day (state after performing the average happiness variation value calculation process on the preceding day). Note that, in FIG. 10, information updated in each process step is underlined.

First, as illustrated in FIG. 9, the control unit 102 decides a process target shop (S202). For example, when deciding the process target shop, the control unit 102 may decide unprocessed shops as the process target by rotation from among shops accumulated in the accumulation unit 104, on the basis of ascending or descending order with regard to the shop IDs.

Next, by using the shop ID of the process target shop, the control unit 102 extracts an entry corresponding to the shop ID in a corresponding time period (current day) from the accumulation unit 104 (S204). Next, the control unit 102 calculates an average value (user average) of happiness variation by using information of the extracted entry (S206). In addition, as shown in the row of the process step S208 in FIG. 10, the control unit 102 adds one to total days in data related to the average happiness variation value of the shop, and adds the user average to the total happiness variation value (S208).

Next, the control unit 102 calculates an average happiness variation value by dividing the total happiness variation value by the total days in FIG. 10, and the control unit 102 updates the average happiness variation value of data related to the average happiness variation value of the shop (S210) as illustrated in the row of the process step S210 in FIG. 10.

In the case where the above described process is completed with regard to all the shops accumulated in the accumulation unit 104 (YES in S212), the average happiness variation value calculation process ends. On the other hand, in the case where there is a shop of which the above describe process is not completed, the process returns to Step S202 and continues.

<3-2. Operation Example of Heat Map Image Generation>

The operation example of the space evaluation made by the space evaluation system 1 has been described above. Next, an operation example of heat map image generation performed by the space evaluation system 1 and the heat map system 2 will be described. Hereinafter, as the operation example of heat map image generation, the operation example of the Happiness Map generation process will be described with reference to FIG. 11, and then the operation example of the my Happiness Map generation process will be described with reference to FIG. 12.

(Happiness Map Generation Process)

FIG. 11 is an explanatory diagram illustrating the operation example of the Happiness Map generation process according to the embodiment. First, as illustrated in FIG. 11, the heat map server 200 decides a group of IDs of shops included in a generated Happiness Map (S302). For example, the shops included in the Happiness Map may be set in advance or may be selected by a user. Alternatively, the shop IDs may be decided on the basis of names, addresses, positions (coordinates) of the shops.

Next, the heat map server 200 transmits one shop ID (space identification information) to the core server 100 (external device) (S304) among the group of shop IDs decided in Step S302. The core server 100 that has received the shop ID transmits (returns) an average happiness variation value (example of evaluation value) of a specific space indicated by the shop ID, to the heat map server 200 (S306).

If average happiness variation values of specific spaces indicated by all the shop IDs in the group of shop IDs decided in Step S302 are not acquired (NO in S308), the processes of Step S304 and 306 are repeated with regard to a shop ID that has not been acquired yet. On the other hand, if the average happiness variation values of the specific spaces indicated by all the shop IDs are acquired (YES in S308), the heat map server 200 decides pixel values in a heat map image on the basis of the average happiness variation values (S310).

Next, the heat map server 200 generates a Happiness Map (heat map image) by using the decided pixel values (S312).

(My Happiness Map Generation Process)

The operation example of the Happiness Map generation process has been described above. Next, an operation example of the My Happiness Map generation process according to the embodiment will be described. FIG. 12 is an explanatory diagram illustrating the operation example of the My Happiness Map generation process according to the embodiment.

First, as illustrated in FIG. 12, the user terminal 220 transmits a group of shop IDs of shops to be included in a My Happiness Map to be generated, and a user ID associated with the user terminal 220 to the heat map server 200 (S402). The way to decide the group of shop IDs of shops to be included in the My Happiness Map to be generated is not limited. For example, the group of shop IDs may be decided on the basis of names, addresses, positions (coordinates) of shops included in a map that the user is currently referring to on a screen.

Next, the heat map server 200 that has received the user ID and the group of the shop IDs transmits the user ID (user identification information) and one shop ID (space identification information) in the group of the shop IDs to the core server 100 (external device) (S404). The core server 100 that has received the user ID and the shop ID transmits happiness variation (example of evaluation value) of a specific user indicated by the user ID with regard to a specific space indicated by the shop ID, to the heat map server 200 (S406).

If happiness variation of the specific user with regard to specific spaces indicated by all the shop IDs in the group of shop IDs are not acquired (NO in S408), the processes of Step S404 and Step 406 are repeated with regard to a shop ID that has not been acquired yet. On the other hand, if the happiness variation of the user with regard to the specific spaces indicated by all the shop IDs are acquired (YES in S408), the heat map server 200 decides pixel values in a heat map image on the basis of the happiness variation (S410).

Next, the heat map server 200 generates a My Happiness Map (heat map image) by using the decided pixel values (S412). The generated My Happiness Map is transmitted from the heat map server 200 to the user terminal 220 (S414), and displayed on the user terminal 220 (S416).

### 4. MODIFIED EXAMPLES

The embodiment of the present disclosure has been described above. Next, some modifications of the embodiment will be described. Note that, the modifications to be described below may be applied to the embodiment separately, or may be applied to the embodiment in combination. In addition, the modifications may be applied instead of the configuration described in the embodiment, or may be applied in addition to the configuration described in the embodiment.

<4-1. First Modification>

In the above described embodiment, the example has been described in which happiness variation (evaluation value) is calculated and accumulated every time a user goes in or out of a specific space. However, the present technology is not limited thereto.

For example, the control unit 102 of the core server 100 does not have to calculate or accumulate happiness variation (evaluation value) in the case where dwell time of a user in a specific space is shorter than predetermined time.

In addition, the control unit 102 of the core server 100 may calculate an evaluation value further on the basis of dwell time of each user in a specific space. For example, the control unit 102 may weight and calculate an evaluation value on the basis of dwell time. Specifically, the evaluation value may be calculated such that a weight of a user with short dwell time is set to be small and a weight of a user with long dwell time is set to be large.

According to such a configuration, it is possible to prevent reduction in accuracy of evaluation values due to calculation and accumulation of happiness variation of a user who has not evaluated sufficiently (whose feeling has not vary sufficiently) since dwell time in the specific space is too short. In addition, according to such a configuration, it is possible to suppress intentional manipulation of evaluation of the specific space by going in and out of the specific space only a short time.

<4-2. Second Modification>

In the above described embodiment, the example has been described in which information regarding happiness variation of a specific user is displayed (notified) as a My Happiness Map on a user terminal of the target user. However, the present technology is not limited thereto. For example, not only a specific user but also a third person may be notified of and use information regarding evaluation values of the specific user such as happiness variation. Specifically, an owner of a specific space (such as a shop) may be notified of evaluation values made by users and information regarding the users.

For example, via the communication unit 206, the control unit 202 of the heat map server 200 may notify an owner of a specific space indicated by space identification information, of identification information of a user corresponding to an evaluation value that satisfies a predetermined condition among users who have gone in and out of the specific space. According to such a configuration, it is possible for the owner to perform efficient information delivery such as recommending a user whose evaluation value of the space satisfies a predetermined condition (such as a condition that happiness level increases by a predetermined value or more) to visit the shop again, for example. In addition, although in general, user experience is evaluated abstractly such as attitudes of staff in a shop, air conditioning, service levels, perspicuity of item presentation, or environment creation, it is possible for the owner to delivery information after evaluating user experience by using indexes based on happiness levels according to the present technology.

Next, an example in which the heat map system 2 cooperates with an SNS and an example in which the heat map system 2 provides an independent service will be described as an example in which information regarding My Happiness Maps is provided to an owner and the owner delivers information.

(Information Delivery in Cooperation with SNS)

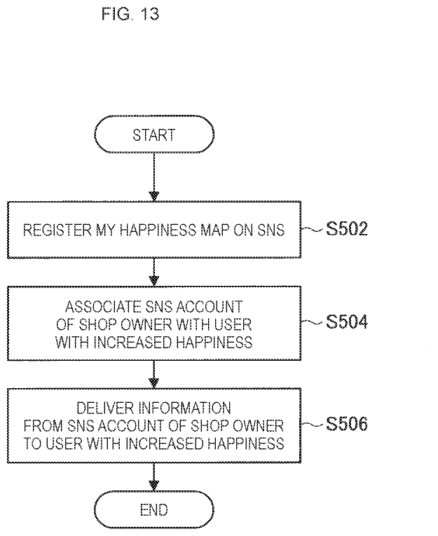

FIG. 13 is an explanatory diagram illustrating an operation example in the case where the heat map system 2 provides information regarding a My Happiness Map to an owner of a space in cooperation with an SNS provided by an external server (SNS server), and the owner delivers information. This operation example has an advantage that a user and an owner of a space who have accounts of the SNS do not have to create new accounts.

First, as illustrated in FIG. 13, a My Happiness Map of a user is registered on (associated with) an account of the user in an SNS (SNS account) (S502). For example, a user may operate a user terminal to register a My Happiness Map on an SNS account of the user after generating the My Happiness Map as described with reference to FIG. 12. Alternatively, the heat map server 200 may provide an application programming interface (API), and an SNS server may obtain access authority through an API approval means such as OAuth to automatically acquire the My Happiness Map.

Next, the SNS account of the shop owner is associated with identification information of a user whose happiness level has increased by a predetermined value or more (user with increased happiness) by going in and out of the shop, and the shop owner is notified thereof (S504). For example, the SNS server may associates the user with the SNS accounts of the shop owners that have been associated in advance with shop IDs included in the My Happiness Map of the user.

Next, information is delivered (information regarding special notification, coupon, or the like is delivered) from the SNS accounts of the shop owners to the user with increased happiness. Such information may be delivered by using a message function or the like provided in the SNS, for example. In addition, such information may be delivered manually by the shop owners or may be delivered automatically through the SNS server.

(Information Delivery Through Independent Service)

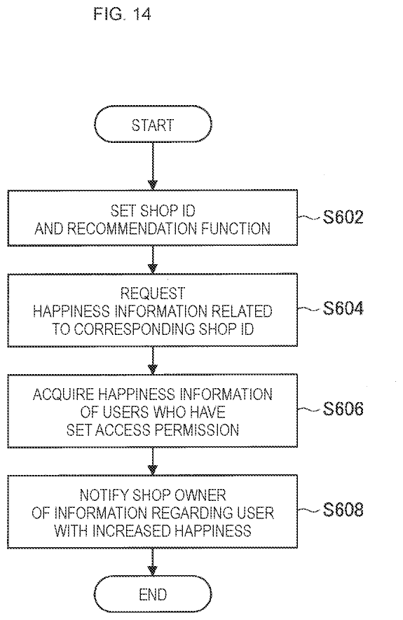

FIG. 14 is an explanatory diagram illustrating an operation example in the case where the heat map system 2 operates as an independent service to provide an information delivery service without cooperating with any SNSs. This operation example has an advantage that a user does not have to register a My Happiness Map on his/her SNS account. Therefore, not only a user who does not want to use the SNS but also a user who does not want to provide information regarding a My Happiness Map to the SNS can use such a service.

First, as illustrated in FIG. 14, a setting (a setting for turning on a function) is configured such that a recommendation (information delivery) function becomes available with regard to each shop ID (S602). Such a setting may be configured manually by a shop owner, or may be configured automatically with respect to a shop ID associated with a cover address (e-mail address or the like) of the shop owner.

Next, the heat map server 200 requests happiness information (happiness variation of users) related to the shop ID from the core server 100 (S604). Here, the heat map server 200 may request happiness information of all users related to the shop ID, or may request happiness information acquired within a limited time period.

Next, the core server 100 determines whether each user has set access permission to provide happiness information of each user to a third person, and transmits happiness information of users who has set the access permission to the heat map server 200 (S606).

The heat map server 200 that has acquired the happiness information notifies the owner of the shop of identification information of a user whose happiness level has increased by a predetermined value or more (user with increased happiness) by going in and out of the shop (S608). Here, the heat map server 200 may notify the owner of the shop of identification information associated in advance with the user ID (personal information such as e-mail address) for delivering information to the user.

<4-3. Third Modification>

In the above described embodiment, the heat map image generation process has been described as an example of information processing using evaluation values of specific spaces. However, the present technology is not limited thereto. For example, the evaluation values of the specific spaces may be used for the recommendation (information delivery) service described in the second modification, or may be used for a ranking service or an application such as a game using position information.

For example, in the present technology, evaluation values may be used for ranking facilities in the same level and the same industry. For example, it is possible to rank restaurants, clothing retail stores, grocery stores, or the like in a shopping mall. In addition to a ranking list of movies based on attendance and reputation, it is possible to provide a new ranking list of movies made by using the evaluation values obtained at the entrance/exit of the movie theater and checking the evaluation values against a movie schedule.

In addition, by using the present technology, it is also possible to introduce a new evaluation index to a gourmet ranking by using evaluation values. In addition, it is possible to provide a gourmet ranking service using evaluation values related to happiness only. In the gourmet ranking service, rankings are made by using various kinds of evaluation methods in general. Therefore, sometimes a ranking separates from direct evaluation made by consumers. It is considered that the happiness level is directly linked to a customer's satisfaction level. Therefore, by using evaluation values related to happiness only, it is possible to provide a ranking related more closely to the direct evaluation made by the consumers.

In addition, according to the present disclosure, it is also possible to rank services of which making a ranking is difficult. For example, it is difficult to rank spaces such as clothing retail stores which are subjectively evaluated by consumers. However, it is considered that evaluation based on happiness is directly affected by inventory status, staff's behavior, and the like in each branch of a chain store. Therefore, it is possible to provide a new ranking to consumers by ranking clothing retail stores or the like on the basis of evaluation values related to happiness. In addition, it is possible to rank retail stores that sell identical items such as supermarkets or convenience stores, by using evaluation values related to happiness. Conventionally, retail stores have been subjectively evaluated by using questionnaires or the like of each stores with regard to staff's service, store layout that makes shopping easier, display that effectively uses lights to emphasize a product, or the like. However, according to the present technology, it is possible to objectively rank retail stores.

In addition, it is possible to apply the present technology not only to business-to-consumer (B2C) services such as the heat map image generation, recommendation, and ranking, but also to business-to-business (B2B) services. For example, information regarding the evaluation values related to happiness may be provided to an owner of the shop. Customer's happiness is linked to evaluation of each shop. Therefore, the customer's happiness serves as an important index for an owner who evaluates store operation to run a business. In such a case, the above described evaluation values according to the embodiment such as happiness variation for each day is important. However, it is necessary to make temporal granularity of evaluation calculation finer in view of evaluation that drastically vary as time advances such as staff's attitudes or the like. For example, the evaluation values may be calculated in a smaller unit such as a semidiurnal unit, a staff time table unit, an hour-basis unit, or the like. In addition, in management of a shop such as a convenience store, evaluation values can serve as a reference for improving the shop by comparing average happiness variation values of respective shops and analyzing a situation of a shop with higher average happiness variation value (such as effects caused by the shop's original strategy or presence or absence of excellent staff) in a certain time slot.

In addition, by using the present technology, it is possible to introduce concept of an evaluation value related to happiness to an application such as a game using position information in a real space. For example, a ranking corresponding to specific spaces may be decided in the game in accordance with evaluation values of the specific spaces.

Such information processing for providing the above described service, application, or the like may be performed by the system 3 or the system 4 described with reference to FIG. 2, for example.

### 5. HARDWARE CONFIGURATION EXAMPLE

The embodiment of the present disclosure and the modifications thereof have been described above. The above described information processing such as the happiness variation accumulation process, the average happiness variation calculation process, and the heat map image generation process is achieved by operating cooperatively software and hardware of the core server 100 or the heat map server 200. Next, a hardware configuration of the information processing device 1000 will be described as hardware configuration examples of the core server 100 and the heat map server 200 that are the information processing devices according to the embodiment.

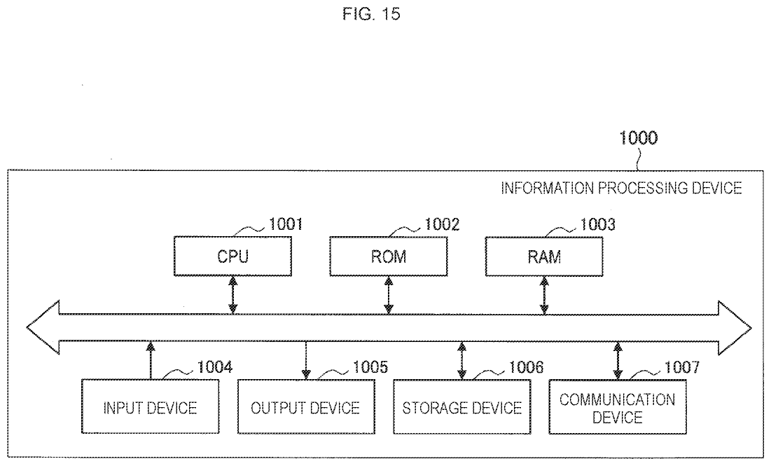

FIG. 15 is an explanatory diagram illustrating a hardware configuration of the information processing apparatus 1000 according to the embodiment. As illustrated in FIG. 15, the information processing device 1000 includes a central processing unit (CPU) 1001, read only memory (ROM) 1002, random access memory (RAM) 1003, an input device 1004, an output device 1005, a storage device 1006, and a communication device 1007.

The CPU 1001 functions as an arithmetic processing device and a control device to control all operation performed in the information processing device 1000 in accordance with various kinds of programs. In addition, the CPU 1001 may be a microprocessor. The ROM 1002 stores programs, operation parameters, and the like used by the CPU 1001. The RAM 1003 transiently stores programs used when the CPU 1001 is executed, and various parameters that change as appropriate when executing such programs. They are connected with each other via the host bus including a CPU bus or the like. Mainly, the functions of the control unit 102 and the control unit 202 are achieved by operating cooperatively software, the CPU 1001, the ROM 1002, and the RAM 1003.

The input device 1004 includes: an input mechanism configured to be used by the user for imputing information, such as a mouse, a keyboard, a touchscreen, a button, a microphone, a switch, or a lever; an input control circuit configured to generate an input signal on the basis of user input and configured to output the signal to the CPU 1001; and the like. By operating the input device 1004, the user of the information processing device 1000 can input various kinds of data into the information processing apparatus 1000 and instruct the information processing apparatus 100 to perform a processing operation.

For example, the output device 1005 includes a display device such as a liquid crystal display (LCD) device, an OLED device, or a lamp. Further, the output device 1005 includes audio output device such as a speaker or headphones. For example, the display device displays captured images, generated images, and the like. On the other hand, the audio output device converts audio data or the like into audio and outputs the audio.

The storage device 1006 is a device for data storage. The storage device 1006 may include a storage medium, a recording device which records data in a storage medium, a reader device which reads data from a storage medium, a deletion device which deletes data recorded in a storage medium, and the like. The storage device 1006 stores therein the programs executed by the CPU 1001 and various kinds of data. The storage device 1006 corresponds to the accumulation unit 104 described with reference to FIG. 3.

The communication device 1007 is a communication interface including, for example, a communication device for connection to a communication network. Further, the communication device 1007 may include a communication device that supports a wireless local area network (LAN), a communication device that supports long term evolution (LTE), a wired communication device that performs wired communication, and a communication device that supports Bluetooth (registered trademark). The communication device 1007 corresponds to the communication unit 106 described with reference to FIG. 3, and the communication unit 206 described with reference to FIG. 4.

Note that, in a way similar to the information processing device 1000, the entrance/exit sensor device 120, the wearable device 140, and the user terminals 220a to 220d each includes hardware equivalent to the CPU 1001, the ROM 1002, the RAM 1003, and the like.

### 6. CONCLUSION

As described above, according to the embodiment of the present disclosure, it is possible to evaluate spaces in association with feelings of users. In addition, it is possible to provide services such as heat map image generation based on space evaluation having a stronger relationship with feelings of users, by using evaluation values obtained by evaluating spaces in association with the feelings of the users.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

For example, the example in which feeling data is a happiness level has been described in the above embodiment. However, the present technology is not limited thereto. For example, the feeling data may be data indicating levels of other kinds of feelings such as sadness or loneliness. In addition, it is possible to provide the above described services by using evaluation values obtained on the basis of the data indicating the levels of the other kinds of feelings.

In addition, in the above described embodiment, another device may be provided with information necessary for the process that is to be performed by a control unit of each device in the space evaluation system 1, and the process may be performed by a control unit of the another device. For example, although the embodiments describe the example in which the control unit 124 of the entrance/exit sensor device 120 acquires feeling data, the entrance/exit sensor device 120 may provide an image to the core server 100, and the control unit 102 of the core server 100 may acquire the feeling data. In addition, in a similar way, a process that is performed by each device in the heat map system 2 may be performed by other devices.

In addition, the embodiment describes the example in which the core server 100 associates a happiness level obtained when a user goes into a space with a happiness level obtained when the user goes out of the space, calculates happiness variation, and calculates an evaluation value on the basis of the happiness variation. However, the present technology is not limited thereto. For example, it is also possible to calculate an evaluation value on the basis of a sum of happiness levels of users who have gone into a space and a sum of happiness levels of the users who have gone out of the space without associating the happiness levels with the users.

In addition, according to the above described embodiment, it is also possible to provide a computer program for causing hardware such as the CPU 1001, ROM 1002, and RAM 1003, to execute functions equivalent to the structural elements of the above described space evaluation system 1 and the heat map system 2. Moreover, it may be possible to provide a recording medium having the computer program stored therein.

In addition, it may not be necessary to chronologically execute respective steps according to the above described embodiment, in the order described in the sequence diagrams or the flow charts. For example, the respective steps in the processes according to the above described embodiment may be processed in the order different from the order described in the sequence diagram or the flow charts, and may also be processed in parallel.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

An information processing system including:

an accumulation unit configured to accumulate variation in feelings of a user caused by going in and out of a specific space; and

a control unit configured to calculate an evaluation value of the specific space on a basis of the variation in the feelings of the user.

(2)

The information processing system according to (1),

in which the control unit calculates the evaluation value on a basis of difference between feeling data obtained when each user goes out of a specific space and feeling data obtained when each user goes into the specific space.

(3)

The information processing system according to (1) or (2),

in which the control unit calculates the evaluation value on a basis of difference between a happiness level obtained when each user goes out of a specific space and a happiness level obtained when each user goes into the specific space.

(4)

The information processing system according to any one of (1) to (3),

in which the control unit does not calculate the evaluation value in a case where dwell time of the user in a specific space is shorter than a predetermined time.

(5)

The information processing system according to any one of (1) to (4),

in which the control unit calculates the evaluation value further on a basis of dwell time of each user in a specific space.

(6)

The information processing system according to any one of (1) to (5),

in which the control unit calculates the evaluation value from feeling data obtained when each user goes into a specific space and feeling data obtained when each user goes out of the specific space, the feeling data being based on information regarding each user detected by a sensor installed such that the sensor is capable of detecting information regarding an entrance/exit of the specific space.

(7)

The information processing system according to any one of (1) to (6),

in which the control unit calculates the evaluation value from feeling data obtained when each user goes into a specific space and feeling data obtained when each user goes out of the specific space, the feeling data being based on biological information of each user detected by a sensor attached to each user when each user goes in and out of the specific space.

(8)

The information processing system according to any one of (1) to (7), including

a communication unit configured to receive space identification information indicating the specific space from an external device,

in which the control unit returns an evaluation value of the specific space indicated by the space identification information to the external device via the communication unit.

(9)

An information processing system including:

a communication unit configured to transmit space identification information indicating a specific space to an external device that is capable of calculating an evaluation value of the specific space, the evaluation value being based on variation in feelings of a user caused by going in and out of the specific space; and

a control unit configured to generate a heat map image by mapping a pixel value representing the evaluation value on a position of the specific space on a basis of the evaluation value of the specific space indicated by the space identification information, the evaluation value being acquired by the external device via the communication unit.

(10)

The information processing system according to (9),

in which the control unit
transmits the space identification information indicating the specific space and user identification information indicating a specific user via the communication unit, and
generates a heat map image for the specific user by mapping a pixel value representing an evaluation value of the specific space on the position of the specific space indicated by the space identification information, on a basis of the evaluation value that is evaluation made by the specific user indicated by the user identification information, the evaluation value being acquired by the external device via the communication unit.

(11)

The information processing system according to (9) or (10),

in which the control unit generates an image in which a pixel value representing an evaluation value corresponding to a first visited space and a pixel value representing an evaluation value corresponding to a second visited space are arranged in chronological order.

(12)

The information processing system according to any one of (9) to (11),

in which, via the communication unit, the control unit notifies an owner of the specific space indicated by the space identification information, of identification information of a user corresponding to an evaluation value that satisfies a predetermined condition among users who have gone in and out of the specific space.

(13)

An information processing method including:

accumulating variation in feelings of a user caused by going in and out of a specific space; and

calculating, by a processor, an evaluation value of the specific space on a basis of the variation in the feelings of the user.

(14)

An information processing method including:

transmitting space identification information indicating a specific space to an external device that is capable of calculating an evaluation value of the specific space, the evaluation value being based on variation in feelings of a user caused by going in and out of the specific space; and

generating, by a processor, a heat map image by mapping a pixel value representing the evaluation value on a position of the specific space on a basis of the evaluation value of the specific space indicated by the space identification information, the evaluation value being acquired by the external device.

### REFERENCE SIGNS LIST

1 space evaluation system
2 heat map system
5 communication network
99 information processing system
100 core server
102 control unit
104 accumulation unit
106 communication unit
120 entrance/exit sensor device
122 communication unit
124 control unit
126 entrance sensor unit
128 exit sensor unit
140 wearable device
142 communication unit
144 control unit
146 sensor unit
160 communication network
200 heat map server
202 control unit
206 communication unit
220 user terminal
260 communication network

What is claimed is:

1. An information processing system, comprising:
a communication unit configured to transmit space identification information indicating a specific space to an external device that is capable of calculating an evaluation value of the specific space, wherein the evaluation value is based on variation in feelings of a user caused by going in and out of the specific space; and
a control unit configured to generate a heat map image by mapping a pixel value representing the evaluation value on a position of the specific space, based on the evaluation value of the specific space, wherein the evaluation value is acquired by the external device via the communication unit.

2. The information processing system according to claim 1, wherein the control unit is further configured to:
transmit the space identification information indicating the specific space and user identification information indicating a specific user via the communication unit; and
generate the heat map image for the specific user by mapping a pixel value representing an evaluation value of the specific space on the position of the specific space, based on the evaluation value that is evaluation made by the specific user indicated by the user identification information, wherein the evaluation value is acquired by the external device via the communication unit.

3. The information processing system according to claim 1, wherein the control unit is further configured to generate an image in which a pixel value representing an evaluation value corresponding to a first visited space and a pixel value representing an evaluation value corresponding to a second visited space are arranged in chronological order.

4. The information processing system according to claim 1,
wherein, via the communication unit, the control unit is further configured to notify an owner of the specific space indicated by the space identification information, of identification information of a user corresponding to an evaluation value that satisfies a predetermined condition among users who have gone in and out of the specific space.

5. An information processing method, comprising:
transmitting space identification information indicating a specific space to an external device that is capable of calculating an evaluation value of the specific space, wherein the evaluation value is based on variation in feelings of a user caused by going in and out of the specific space; and
generating a heat map image by mapping a pixel value representing the evaluation value on a position of the specific space, based on the evaluation value of the specific space indicated by the space identification information, wherein the evaluation value is acquired by the external device.
